(19) United States
(12) Patent Application Publication
(10) Pub. No.: US 2026/0157694 A1
(43) Pub. Date: Jun. 11, 2026

(54) Eye Massaging Device with EEG Measurement and Adaptive Massage Functionality

(71) Applicants: David Yang, Santa Ana, CA (US); Rene Jon Hart, New Canaan, CT (US)

(72) Inventors: David Yang, Santa Ana, CA (US); Rene Jon Hart, New Canaan, CT (US)

(21) Appl. No.: 19/410,898

(22) Filed: Dec. 5, 2025

Related U.S. Application Data
(60) Provisional application No. 63/729,146, filed on Dec. 6, 2024.

(51) Int. Cl. A61B 5/00; A61B 5/16; A61B 5/256; A61B 5/265; A61B 5/27; A61B 5/372; A61H 9/00; A61H 23/02; A61M 21/00; A61M 21/02

(52) U.S. Cl. CPC A61B 5/4836; A61B 5/165; A61B 5/256; A61B 5/265; A61B 5/27; A61B 5/372; A61B 5/4809; A61B 5/6803; A61B 5/7264; A61B 5/7475; A61H 9/0007; A61H 9/005; A61H 23/02; A61M 21/02; A61B 2560/0468; A61B 2562/0215; A61B 2562/227; A61H 2201/0107; A61H 2201/0207; A61H 2201/0214; A61H 2201/10; A61H 2201/1238; A61H 2201/1607; A61H 2201/165; A61H 2201/5007; A61H 2201/5023; A61H 2201/5097; A61H 2205/024; A61H 2230/105; A61M 2021/0022; A61M 2021/0066; A61M 2021/0072; A61M 2205/0233; A61M 2205/054; A61M 2205/3303; A61M 2205/3592; A61M 2205/3606; A61M 2205/502; A61M 2209/088; A61M 2210/0606; A61M 2210/0612; A61M 2230/005; A61M 2230/10

(57) ABSTRACT

Wearable eye massaging device is disclosed, incorporating electroencephalogram (EEG) sensors and adaptive control logic to dynamically adjust massage parameters based on the user's brain wave activity. The device includes one or more EEG sensing elements integrated into the wearable structure, each comprising a conductive textile electrode, a compressible foam backing layer, and a mechanical crimp or clamp for signal connection. The massage system may include heat, cooling, vibration, pneumatic compression, and/or EMS (electrical muscle stimulation), which are selectively activated or modulated in response to EEG-derived indicators of mental states such as stress, relaxation, focus, or sleep readiness. The device operates independently or in communication with external processing units or companion applications. Optional features include user-specified mental state targeting, real-time program adaptation, sleep-safe operation, session wind-down, and cross-device coordination. The system provides a personalized, EEG-responsive wellness experience through closed-loop biofeedback control integrated into a compact, comfortable, and flexible eye-mounted device.

### TECHNICAL FIELD OF THE INVENTION

The present invention relates to personal wellness and neuroadaptive therapeutic devices. More particularly, it concerns a wearable eye massaging device equipped with electroencephalogram (EEG) sensors for monitoring brain wave activity and adaptively controlling massage parameters in real time to support user-specific mental states such as relaxation, focus, or sleep.

### BACKGROUND OF THE INVENTION

Wearable eye massagers are commonly used to relieve ocular strain, improve circulation around the eyes, and promote general relaxation. These devices often incorporate features such as heat, vibration, pneumatic compression, and electrical muscle stimulation (EMS). While beneficial, most commercially available eye massagers rely on pre-set programs or limited user input and cannot adjust their operation in response to the user's real-time physiological or cognitive state.

Some prior art devices attempt to personalize user experience by integrating heart rate monitors or motion sensors. However, such inputs do not provide sufficient resolution to detect cognitive or emotional states such as heightened stress, mental fatigue, relaxation readiness, or sleep transitions.

Electroencephalogram (EEG) sensing, which detects electrical activity in the brain, offers a more direct and detailed method for assessing a user's mental state. Yet, EEG sensing has traditionally required rigid electrodes, conductive gels, or stationary settings, limiting its practical integration into consumer wellness devices, especially those involving mechanical actuation or facial contact.

Accordingly, there remains a need for an eye-mounted wellness device that combines EEG sensing with adaptive massage functionality in a compact, wearable form. Such a device should be capable of dynamically adjusting massage parameters based on real-time brain wave data while maintaining comfort, signal integrity, and safety during operation. The present invention addresses these and other limitations of the prior art.

### SUMMARY OF THE INVENTION

The present invention provides a wearable eye massaging device that integrates electroencephalogram (EEG) sensing with real-time adaptive control of massage parameters to deliver a personalized, neuroresponsive wellness experience. The device is configured to detect brain wave activity through one or more EEG sensing elements embedded within the wearable structure, and to dynamically adjust massage outputs based on the user's cognitive or emotional state.

In certain embodiments, the EEG sensing element comprises a multi-layer construction including a skin-contacting conductive textile electrode, a compressible foam backing layer, and a mechanical crimp or clamp connecting the signal wire to the textile. This configuration maintains stable signal acquisition during massage operations involving vibration, pneumatic compression, or thermal cycling, while preserving user comfort.

The massage mechanism may include one or more of heat, cooling, vibration, pneumatic compression, and EMS. These outputs may be modulated automatically in response to real-time EEG data to promote states such as relaxation, focus, or sleep readiness. In some embodiments, the system includes a user interface for selecting a target mental state, and a closed-loop feedback engine that adjusts sensory output based on deviations from that target.

Optional features include EEG-driven session wind-down protocols, safety adaptations during sleep onset, EEG-triggered wake-up routines, migraine intervention modes, and coordination with external wellness or environmental devices. The system may process EEG data locally or transmit it to an external device or application for cloud-based analysis and control.

Through its integration of wearable EEG sensing, adaptive massage control, and user-specific program customization, the invention enables a highly personalized and responsive therapeutic experience not found in prior art wellness devices.

### REFERENCE CHARACTERS AND PARTS LIST

10—Eye massaging device (overall assembly)
12—Outer wearable structure (mask body)
14—EEG sensing element/EEG sensor assembly
16—Conductive textile electrode (skin-contacting EEG sensor layer)
18—Compressible foam backing layer (behind conductive textile)
20—Reinforcement layer (optional; prevents tearing at crimp junction)
22—Crimp or mechanical clamp (electrical connection from textile to signal wire)
24—Signal wire/electrical lead connected to EEG processing circuit
26—Massage mechanism (schematic; may include vibration, compression, heat, etc.)
28—Pneumatic chamber (optional massage element)
30—Vibration motor (optional massage element)
32—Heating/cooling element
34—Onboard EEG processor or controller (for real-time analysis and actuation)
36—Power supply or battery
38—Wireless communication module (e.g., Bluetooth®, Wi-Fi)
40—External device (e.g., smartphone, tablet, cloud server) for remote EEG data processing
42—Sensor pocket or mounting aperture in mask interior
44—User interface/control panel/mobile app interface

### BRIEF DESCRIPTION OF THE DRAWINGS

The accompanying drawings, which are incorporated herein and form a part of the specification, illustrate the present invention and, together with the description, further serve to explain the principles of the invention and to enable a person skilled in the pertinent art to make and use the invention.

FIG. 1 is a front elevation view of the wearable eye massaging device, showing the overall outer shell contour and general shape configured to fit the user's face.

FIG. 2 is a rear interior view of the device, illustrating the arrangement of internal components, including EEG sensing elements and massage actuator locations positioned for contact with the peri-orbital regions.

FIG. 3 is a partial cross-sectional side view of the device, showing the multi-layer EEG sensing element integrated into the interior surface. The section reveals the conductive textile electrode, compressible foam backing, and signal wire crimp interface.

FIG. 4 is a schematic diagram of the device's electrical and functional components, illustrating the communication between EEG sensors, the massage control unit, optional wireless modules, and external processing devices.

FIG. 5 is an exploded perspective view of the EEG sensor assembly, showing the layered construction comprising a conductive textile electrode, compressible foam layer, optional reinforcement layer, and mechanical crimp connection to a signal lead.

### DETAILED DESCRIPTION OF THE INVENTION

In the following detailed description of the invention of exemplary embodiments of the invention, reference is made to the accompanying drawings (where like numbers represent like elements), which form a part hereof, and in which is shown by way of illustration specific exemplary embodiments in which the invention may be practiced. These embodiments are described in sufficient detail to enable those skilled in the art to practice the invention, but other embodiments may be utilized; and logical, mechanical, electrical, and other changes may be made without departing from the scope of the present invention. The following detailed description is, therefore, not to be taken in a limiting sense, and the scope of the present invention is defined only by the appended claims.

In the following description, numerous specific details are set forth to provide a thorough understanding of the invention. However, it is understood that the invention may be practiced without these specific details. In other instances, well-known structures and techniques known to one of ordinary skill in the art have not been shown in detail in order not to obscure the invention. Referring to the figures, it is possible to see the various major elements constituting the apparatus of the present invention.

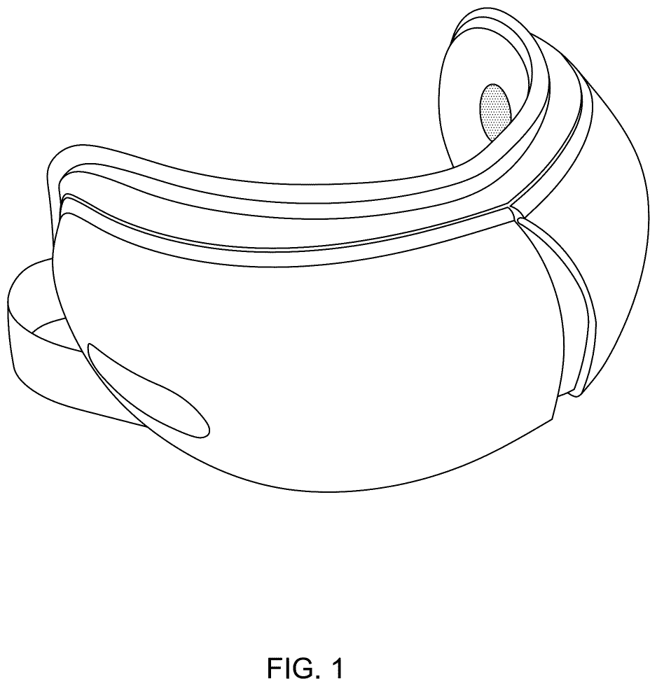

Referring to FIGS. 1-5, the present invention discloses a wearable eye massaging device (10) configured to provide personalized therapeutic output based on real-time measurements of brain wave activity. The device integrates an EEG sensing system (14) into a soft, comfortable mask-like structure (12) worn over the eyes, allowing continuous monitoring of the user's cognitive state while delivering adjustable massage stimulation.

### Device Overview and External Form

As shown in FIG. 1, the eye massaging device (10) comprises an ergonomically contoured outer housing (12) designed to comfortably fit around a user's eyes and upper face. The outer structure is made of flexible, skin-safe materials and is shaped to conform to facial anatomy while securely retaining internal components. The wearable form factor allows hands-free operation, supporting relaxation, focus, and sleep use cases.

### Rear Interior View and Component Placement

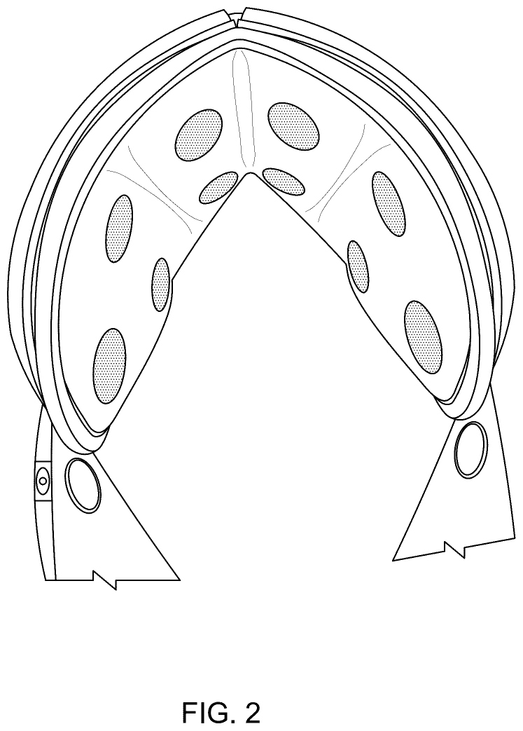

FIG. 2 illustrates the interior surface of the device as it would contact the user's face. Within this interior region, multiple EEG sensing elements (14) are embedded at selected anatomical positions such as the forehead, temples, or peri-orbital regions. Also visible are massage actuator components including pneumatic chambers (28), vibration motors (30), and thermal elements (32) arranged to provide targeted stimulation.

The EEG sensing elements are positioned to ensure consistent electrical coupling with the skin, even during massage operation, while massage actuators are aligned to avoid obstruction of sensor performance.

### EEG Sensor Construction

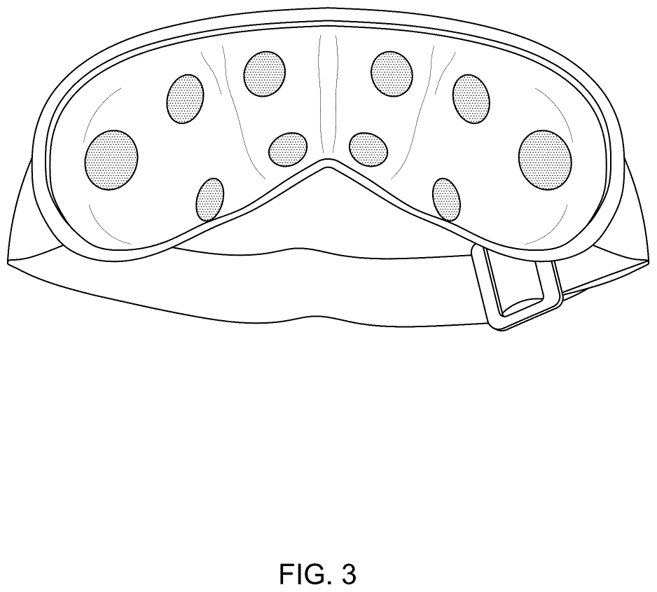

FIG. 3 provides a cross-sectional view of the multi-layer EEG sensor (14). Each sensor comprises:

A conductive textile electrode (16) configured to contact the user's skin. This textile may include silver-coated nylon or polyester, conductive mesh, or other soft, low-impedance materials optimized for biopotential acquisition.

An optional compressible foam backing layer (18) may be positioned directly behind the textile. When included, this foam structure helps maintain gentle pressure against the skin, promotes conformal contact, and isolates the electrode from massage vibrations or compression forces. Suitable materials include memory foam, silicone foam, EVA, or open-cell polyurethane.

In certain embodiments, the electrode assembly may omit the foam backing layer entirely, relying instead on the inherent flexibility of the textile and crimped conductor to maintain adequate skin conformity. Even without the foam structure, the electrode can still acquire biopotential signals with sufficient fidelity for EEG monitoring, provided that the textile maintains consistent skin engagement during normal use.

When a foam backing layer is employed, its thickness, density, and compressive modulus may be selected to balance comfort with signal quality. In other embodiments, the electrode may include only a minimal or low-density spacer layer, or a gel-infused textile, each serving as alternative structures for promoting uniform pressure distribution without requiring a dedicated foam component.

An optional reinforcement layer (20) located at the rear side of the foam. This structural layer provides tear resistance and strengthens the textile-crimp interface.

A mechanical crimp or clamp (22) securely connects a signal wire (24) to the conductive textile without soldering. The crimp interface ensures reliable electrical continuity and structural stability under dynamic conditions.

This layered construction forms a raised sensing surface that protrudes slightly from the mask interior, as shown in FIG. 3, providing secure yet comfortable contact during device use. The foam damping effect minimizes motion artifacts and maintains signal quality even during operation of the massage system.

### Exploded Sensor View

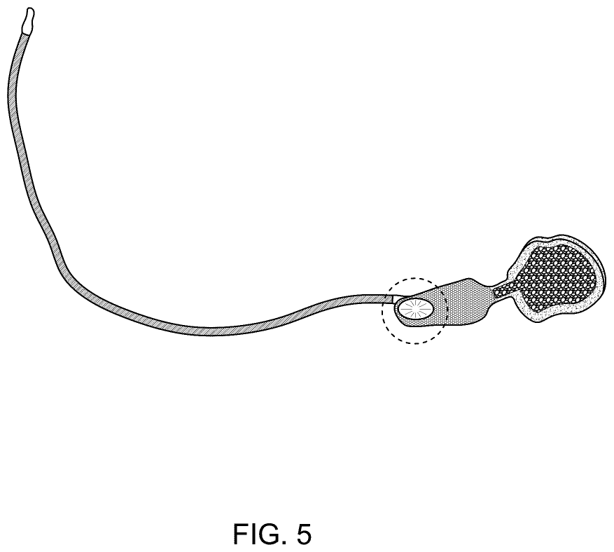

The full sensor stack is shown in FIG. 5, illustrating the exploded configuration of the conductive textile (16), foam layer (18), reinforcement layer (20), and crimp terminal (22) connected to the signal wire (24). This figure emphasizes the modularity and manufacturability of the sensor design. In some embodiments, the EEG sensing elements may be modular, removable, or replaceable to facilitate hygiene and maintenance.

### Functional System Architecture

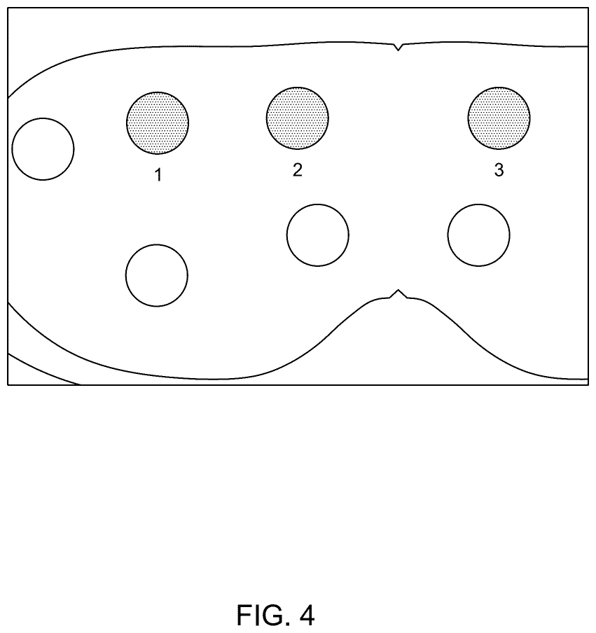

FIG. 4 presents a schematic view of the functional system architecture. The EEG sensor array (14) is electrically coupled to an onboard processing module (34). This processor may analyze brain wave signals locally using embedded firmware, machine learning models, or heuristic pattern analysis. In alternative embodiments, raw or pre-processed EEG data is transmitted wirelessly via communication module (38) to an external device (40)—such as a smartphone, tablet, or cloud service—for analysis and remote control.

The processor outputs real-time control signals to the massage mechanism (26), which may include one or more of: Pneumatic chambers (28) for rhythmic compression; Vibration motors (30) for localized stimulation; Heating or cooling elements (32) for thermal therapy; EMS electrodes (not shown) for muscle stimulation.

### Adaptive Operation and Program Logic

The device operates in a closed-loop feedback mode, where EEG-derived signals are continuously or periodically analyzed to determine the user's mental state. Based on this analysis, massage outputs are adjusted to promote a target state such as deep relaxation, focus, or sleep.

In some embodiments, the user may designate a target mental state via a control panel or user interface (44). The processor compares the real-time EEG-derived state to this target and modifies massage parameters to minimize the difference, using variable intensity, mode switching, and output sequencing.

For example, elevated beta activity may trigger calming massage patterns. Increased delta or alpha waves may prompt a sleep-supportive wind-down protocol. Detection of migraine-like EEG signatures may initiate cold compression, vibration suppression, or, in some embodiments, targeted electrical muscle stimulation (EMS) protocols to help mitigate migraine-associated tension or discomfort.

In some embodiments, the system may incorporate optional electrical muscle stimulation (EMS) circuitry configured to deliver low-level therapeutic pulses to periorbital or temporalis regions. These EMS outputs may be triggered based on EEG-derived indicators of muscular tension, fatigue, or migraine onset patterns. The EMS modality is not required for normal operation of the mask but may be included as an auxiliary therapeutic pathway for users who benefit from neuromuscular relaxation techniques.

The EMS protocol, when present, may be dynamically modulated according to the user's ongoing neural signals. For example, increased detection of stress-correlated EEG signatures may result in gentle EMS pulses designed to relax periocular muscles, while more pronounced migraine-like patterns may initiate a brief targeted EMS routine coordinated with cooling or compression elements.

### Session Wind-Down and Sleep-Safe Features

Upon detecting that the user has entered a restful or sleep state, the device may automatically initiate a wind-down routine, gradually reducing massage intensity and audio output to avoid disruption. In sleep-safe modes, the processor may suspend or modulate mechanical outputs to ensure continued comfort and safety during extended wear.

Environmental and Cross-Device Integration

In certain embodiments, the device may communicate with external wellness devices or smart home systems. For instance, EEG readings may be used to control lighting, ambient sound, or temperature through external signals transmitted via the wireless module (38).

Environmental context—such as ambient noise levels, room lighting conditions, aroma or scent profiles delivered by integrated or external diffusers, and time of day—may also be factored into the adaptive program logic to enhance the effectiveness of the session. In some embodiments, the system monitors or receives inputs regarding these environmental parameters and dynamically adjusts stimulation patterns, relaxation sequences, or user-interface cues to maintain an optimal therapeutic environment. By incorporating controllable sensory cues, including automated or user-selected aromatic outputs, the system can further personalize the session to support relaxation, focus, or other desired physiological or psychological outcomes.

In certain embodiments, the system may interface with an external aroma diffuser or include an integrated scent-release module capable of emitting calming, energizing, or therapeutic aromatic compounds. The selection and timing of aroma delivery may be determined by the adaptive program logic, which evaluates the user's EEG state, environmental conditions, or session objectives.

The aromatherapy component, when available, may operate as a non-contact sensory cue that complements tactile and visual stimuli. For instance, upon detecting EEG patterns associated with restlessness or difficulty transitioning into relaxation, the system may trigger the controlled release of a mild lavender or chamomile scent to support parasympathetic activation. Conversely, focus-supporting aromas may be delivered during cognitive-training or alertness-oriented sessions.

### Independent EEG Operation

In some configurations, the EEG module may function independently of the massage mechanism. This allows the wearable to serve as a standalone EEG sensing device for purposes such as sleep tracking, stress monitoring, or cognitive state detection, even when massage features are inactive.

The present invention thus provides a compact, wearable, EEG-responsive eye massaging system that combines robust signal acquisition with dynamic, state-dependent actuation. Through its innovative sensor construction, real-time feedback control, and user-specific program adaptability, the device delivers a highly personalized and effective therapeutic experience not achievable with static or preprogrammed massagers.

Thus, it is appreciated that the optimum dimensional relationships for the parts of the invention, to include variation in size, materials, shape, form, function, and manner of operation, assembly, and use, are deemed readily apparent and obvious to one of ordinary skill in the art, and all equivalent relationships to those illustrated in the drawings and described in the above description are intended to be encompassed by the present invention.

Furthermore, other areas of art may benefit from this method and adjustments to the design are anticipated. Thus, the scope of the invention should be determined by the appended claims and their legal equivalents, rather than by the examples given.

1. A wearable eye massaging device comprising:
a wearable housing configured to cover a peri-orbital region of a user's face;
at least one EEG sensing element integrated into an interior surface of the housing, each EEG sensing element comprising:
a conductive textile electrode configured to contact the user's skin,
an optional compressible foam backing layer disposed behind the conductive textile electrode, and
a mechanical crimp or clamp connecting a signal wire to the conductive textile electrode;
a massage mechanism configured to apply one or more forms of sensory stimulation, including heat, cooling, vibration, pneumatic compression, and optionally electrical muscle stimulation (EMS);
a control circuit configured to receive EEG signals from the EEG sensing element, analyze the EEG signals to determine a mental state of the user, and dynamically adjust one or more parameters of the massage mechanism in response to the determined mental state.

2. The device of claim 1, wherein the EEG sensing element includes a reinforcement layer positioned between the foam backing layer and the crimp to prevent tearing of the textile electrode.

3. The device of claim 1, wherein the control circuit includes a processor configured to classify the user's mental state as one of: relaxed, alert, focused, drowsy, or stressed.

4. The device of claim 1, wherein the massage mechanism includes a pneumatic compression actuator positioned to apply rhythmic pressure to the user's temples or eye area.

5. The device of claim 1, further comprising a wireless communication module configured to transmit EEG data to an external computing device for processing or visualization.

6. The device of claim 1, further comprising a user interface allowing the user to specify a target mental state, wherein the control circuit modifies the massage mechanism to reduce a difference between the target state and the user's detected state.

7. The device of claim 1, wherein the control circuit is configured to initiate a wind-down sequence by gradually decreasing massage intensity upon detecting a transition to a sleep-like EEG pattern.

8. The device of claim 1, wherein the EEG sensing element is modular and removably mounted within the wearable housing.

9. The device of claim 1, wherein the conductive textile electrode is selected from the group consisting of: silver-coated nylon, conductive mesh, or metal-infused knit fabric.

10. The device of claim 1, wherein the foam backing layer has a thickness between 1 mm and 8 mm and a compressive response between 0.5 N/cm² and 3.0 N/cm².

11. A method of providing EEG-responsive eye massage therapy, comprising:
positioning a wearable eye massaging device over a user's eyes, the device comprising at least one EEG sensing element and a massage mechanism;
detecting brain wave activity using the EEG sensing element, the EEG sensing element comprising a conductive textile electrode in contact with the user's skin and optionally a compressible foam backing layer;
analyzing the detected brain wave activity to determine a cognitive or emotional state of the user; and
automatically adjusting at least one operational parameter of the massage mechanism based on the determined state to promote relaxation, focus, sleep readiness, or stress reduction.

12. The method of claim 11, further comprising receiving user input selecting a target brain state and adjusting the massage mechanism to guide the user toward the target state.

13. The method of claim 11, wherein analyzing the brain wave activity comprises applying a machine learning model to classify EEG signals in real time.

14. The method of claim 11, wherein adjusting the massage mechanism includes modulating a thermal output based on EEG-derived indicators of stress or relaxation.

15. The method of claim 11, wherein the EEG sensing element comprises a layered structure that isolates the electrode from massage-induced mechanical forces.

16. The method of claim 11, further comprising transmitting EEG data wirelessly to a mobile application for display, control, or session tracking.

17. A wearable EEG sensing module comprising:
a flexible sensor assembly comprising a conductive textile electrode layer configured to contact human skin,
am optional compressible foam backing layer configured to apply gentle pressure to maintain contact with the skin, and
a mechanical crimp or clamp connecting a signal lead to the conductive textile electrode;
wherein the sensor assembly is configured for integration into a wearable therapeutic device and is operable to acquire EEG signals during dynamic physical conditions including vibration or compression.

18. The EEG sensing module of claim 17, wherein the compressible foam backing layer includes a dual-density configuration with a soft outer surface and a stiffer interior.

19. The EEG sensing module of claim 17, wherein the crimp or clamp is positioned on the foam side of the assembly to avoid contact with the user's skin.

20. The EEG sensing module of claim 17, further comprising a removable mounting interface for installation within a mask, headband, or other wearable therapeutic device.
